(19) United States
(12) Patent Application Publication
(10) Pub. No.: US 2026/0056574 A1
(43) Pub. Date: Feb. 26, 2026

(54) System with inbuilt mobile cellular communication capability and body vital measuring capability wearable on a human finger

(71) Applicant: Corey Kain, Angola, IN (US)

(72) Inventor: Corey Kain, Angola, IN (US)

(21) Appl. No.: 19/017,881

(22) Filed: Jan. 13, 2025

Related U.S. Application Data
(60) Provisional application No. 63/685,153, filed on Aug. 20, 2024.

(51) Int. Cl. G06F 1/16; A44C 9/00; A61B 5/00; G16H 10/65; H04W 64/00

(52) U.S. Cl. CPC G06F 1/1613; A61B 5/6826; G16H 10/65; H04W 64/003; A44C 9/0053

(57) ABSTRACT

The present invention relates to a wearable communication smart device in the form of ring which provides two way communication system through wireless and cellular networks. The smart device comprises of physical body and a software functionality built for smooth running of the smart device.

### CROSS REFERENCE TO RELATED APPLICATION

The present application is a continuation of U.S. Provisional Patent Application No. 63/685,153 filed on 20 Aug. 2024, the contents of which is incorporated herein by reference.

### BACKGROUND

The concept of smart rings traces its origins to the early developments in miniaturized computing and wearable technology during the late 1990s and early 2000s. The first patents for ring-based electronic devices appeared in the late 1990s, primarily focusing on basic data storage and display capabilities. However, the true evolution of smart rings as we know them today began in the early 2010s, driven by advancements in miniaturization of electronic components and the growing demand for unobtrusive wearable technology.

The initial breakthrough came in 2011 when researchers at MIT developed prototype rings capable of reading printed text for visually impaired users, demonstrating the potential for complex electronics in a ring form factor. This was followed by the first commercially-oriented smart ring projects in 2013, which introduced NFC-enabled rings for contactless payments and digital access control. These early devices, while revolutionary in concept, were limited to passive functionality and required no power source.

The period between 2013 and 2015 marked a significant transition as the first generation of true “smart rings” emerged. These devices incorporated Bluetooth Low Energy (BLE) technology, small batteries, and basic notification systems. Companies like Ringly and MOTA introduced rings that could vibrate or illuminate to alert users of smartphone notifications. However, these devices faced substantial challenges in battery life and user interface design due to their small form factor.

The period of 2016 to 2018 saw the introduction of more sophisticated sensors and improved power management systems. Manufacturers began incorporating accelerometers and gyroscopes, enabling gesture control and basic activity tracking. The Oura Ring, launched in 2018, represented a major advancement by successfully integrating complex health monitoring sensors into a ring form factor, including temperature sensors and photoplethysmography (PPG) for heart rate monitoring.

During 2019-2021, smart rings evolved to include more advanced health monitoring capabilities:
Heart Rate Variability (HRV) monitoring
Sleep pattern analysis
Blood oxygen level estimation
Stress monitoring
Temperature tracking

Thereafter, smart ring seeks more attention by the companies like apple® Samsung® and other mainstream companies who have started adding more and more options in smart rings in daily lives but all of the earlier disclosures and these advancements continued to rely heavily on smartphone connectivity through Bluetooth and NFC protocols, creating significant limitations in functionality and real-time monitoring capabilities. The dependence on proximate smartphone connectivity has remained a critical constraint, particularly in emergency situations or when continuous health monitoring is essential.

Current market solutions (2022-2024) have made incremental improvements in battery life, sensor accuracy, and design aesthetics. However, they continue to operate within the fundamental constraints of short-range wireless communications.

### Notable Limitations Include

1. Dependency on smartphone proximity for data transmission
2. Inability to function autonomously for extended periods
3. Limited real-time monitoring and alert capabilities
4. Absence of direct emergency communication features
5. Restricted range of operation

The present invention, GUARDIAN, addresses these limitations by integrating cellular connectivity through nano-SIM/eSIM technology, thereby revolutionizing the capabilities of smart ring devices. Furthermore, GUARDIAN incorporates advanced artificial intelligence capabilities, setting it apart from existing solutions. The AI system enables intelligent voice processing for natural communication, predictive health analytics for early warning of potential health issues, and adaptive learning of user behavior patterns for personalized responses. The AI engine processes sensor data in real-time to identify anomalies, automate emergency responses, and optimize power consumption based on usage patterns. Additionally, the system employs AI-driven voice synthesis for clear audio output despite the device's compact size, and machine learning algorithms for gesture recognition and contextual awareness.

This innovation enables direct two-way communication and autonomous operation, marking a significant advancement in wearable technology. By combining cellular connectivity, AI capabilities, and advanced health monitoring sensors in a compact ring form factor, GUARDIAN represents the next evolution in smart ring technology, offering unprecedented independence, intelligence, and functionality for users.

### SUMMARY OF INVENTION

The present invention introduces GUARDIAN (Geofence-triggered, Unique, Adjustable Ring Data-syncing Infrared-sensitive-vitals and Activated with Nano-sim/eSIM communication facility), an advanced wearable device that revolutionizes the smart ring category through its innovative integration of cellular communication capabilities and comprehensive health monitoring features. This invention represents a significant departure from traditional smart ring designs by incorporating both nano-SIM and eSIM technologies, enabling direct cellular connectivity without requiring proximity to a smartphone or other intermediary device.

The GUARDIAN system's primary innovation lies in its ability to provide autonomous two-way mobile communication capabilities within a compact ring form factor. The device incorporates an embedded speaker and microphone system, enabling users to initiate and receive voice calls directly through the ring. This functionality is activated through an intuitive tap/push mechanism integrated into the ring's surface, allowing for easy interaction even in emergency situations. The communication system operates independently of traditional Bluetooth or NFC connections, marking a significant advancement over existing smart ring technologies that require constant smartphone connectivity.

In terms of health monitoring capabilities, GUARDIAN implements a sophisticated array of sensors for continuous vital sign tracking. The system monitors critical health parameters including Heart Rate Variability (HRV), blood oxygen saturation (SPO2), stress levels, and sleep patterns. Additionally, the device incorporates fall detection technology, making it particularly valuable for elderly users or those requiring continuous health monitoring. These health monitoring features operate continuously and can trigger automatic alerts when abnormal patterns are detected, providing an essential safety net for users.

The physical design of GUARDIAN prioritizes both functionality and user comfort. Despite its comprehensive feature set, the device maintains a weightless profile and standard ring dimensions. The ring's structure incorporates an adjustable design that enables one size to fit all users, addressing a common limitation in wearable devices. The entire system is housed within a waterproof enclosure, ensuring durability and continuous operation in various environmental conditions. The device's power management system provides extended battery life with efficient standby capabilities, crucial for a device intended for continuous wear.

GUARDIAN's intelligent features extend beyond basic communication and health monitoring. The system incorporates geofence-triggered functionality, allowing for automated responses based on the user's location. The device utilizes AI-based voice processing for natural interaction during calls and notifications, while also supporting gesture-based controls for various functions. The location tracking capability provides real-time positioning data, essential for both everyday use and emergency situations.

Data management and synchronization form a crucial component of the GUARDIAN system. The device implements real-time data synchronization for vital signs and other metrics, transmitting information directly to cloud-based servers through its cellular connection. This data is accessible through a companion mobile application, which serves as a comprehensive management interface for the system. The application enables users to configure notification preferences, manage contact information, view location tracking history, and access complete data logs of all ring events.

The invention is further enhanced by its dynamic notification system, which can intelligently alert users and their designated contacts based on configurable triggers and thresholds. This feature, combined with the device's ability to maintain constant cellular connectivity, ensures that critical information and alerts are delivered promptly, regardless of the user's proximity to their smartphone. The system's architecture supports both preset and customizable notification parameters, allowing for personalized monitoring and alert configurations.

Through this comprehensive integration of communication technology, health monitoring capabilities, and intelligent features within a wearable ring form factor, GUARDIAN represents a significant advancement in personal health and safety devices. Its ability to function autonomously while providing continuous monitoring and communication capabilities addresses critical limitations in existing wearable technology, particularly in emergency situations where immediate communication and health data access are essential.

### TECHNICAL SPECIFICATIONS

### 1. Physical Dimensions and Construction

### Ring Body:

Width: 14-15 mm
Base Thickness: 7 mm
Friction: 2 mm
Touch Button: 7×7×9 mm
Overall Ring Diameter: Variable (adjustable design)

### Material Composition:

Elastic, water-resistant outer shell
Bio-compatible inner surface
Impact-resistant housing for electronic components
Sealed waterproof construction

### 2. Core Component Architecture (Reference Numbers From Technical Drawings)

### A. Communication Module (100 Series)

Nano-SIM/eSIM Module (100)
Integrated Speaker Unit (110)
High-sensitivity Microphone (120)
Push/Tap Interface (130)

### B. Sensor Array (200 Series)

Heart Rate Sensor Module (200)
G-Sensor/Accelerometer (210)
IR Sensor/Receiver (220)
Fall Detection System Integration

### C. Core Systems (300-500 Series)

Power Management Unit (300)
Central Microprocessor (400)
Waterproof Elastic Body (500)

### 3. Sensor Specifications and Capabilities

### Heart Rate Monitoring:

Continuous measurement capability
Heart Rate Variability (HRV) analysis
Real-time data processing

### Blood Oxygen (SPO2) Monitoring:

Infrared-based measurement
Continuous tracking capability

### Motion and Position Detection:

3-axis accelerometer
Gesture recognition capability
Fall detection algorithms

### 4. Communication Systems

### Cellular Connectivity:

Nano-SIM/eSIM support
Voice call capability
Data transmission

### Location Services:

GPS/GNSS tracking
Geofence capability
Real-time location updates

### 5. Power Management

### Battery System:

Extended standby time
Quick charge capability
Power optimization algorithms

### Energy Efficiency:

Smart power management
Activity-based power adjustment
Low-power standby mode

### 6. Data Processing and Storage

### Local Processing:

Real-time sensor data analysis
Emergency detection algorithms
Voice processing

### Cloud Integration:

Secure data transmission
Real-time synchronization
Historical data storage

### 7. Environmental and Safety Specifications

Water Resistance: IP68 rated
Operating Temperature Range: −10° C. to 45° C.
Impact Resistance: Standard drop test compliant
EMI/EMC: Compliant with international standards
SAR Compliance: Within safety limits for wearable devices

### 8. Interface and Control Systems

### Touch Interface:

Capacitive touch surface
Multi-gesture support
Pressure-sensitive triggering

### Voice Interface:

AI-powered voice recognition
Natural language processing
Voice command support

### 9. Application Integration

### Mobile App Capabilities:

Real-time monitoring
Historical data analysis
Settings configuration

### Emergency Contact Management

### Cloud Platform:

Secure data storage
Analytics engine
User profile management
Device configuration

### 10. Advanced Features

### AI Implementation:

Predictive health analytics
Behavioral pattern recognition
Automated emergency response

### Security Features:

End-to-end encryption
Secure boot
Tamper detection

### Emergency Response:

Automated SOS triggering
Location broadcasting
Emergency contact notification
   These technical specifications are designed to ensure optimal performance while maintaining the compact form factor of a wearable ring device. The integration of these components is achieved through advanced miniaturization techniques and efficient space utilization within the ring's structure.

### BRIEF DESCRIPTION OF THE DRAWINGS

FIG. 1 illustrates a basic diagram of the GUARDIAN smart ring showing the primary component layout in a circular arrangement according to an embodiment of the present invention.

FIG. 2 depicts a cross-sectional view of the GUARDIAN smart ring with components and enclosure details according to an embodiment of the present invention.

FIG. 3 presents a top view of the GUARDIAN smart ring with enclosure housing components inside according to an embodiment of the present invention.

FIG. 4 shows a detailed diagram of the GUARDIAN smart ring with components housed and interconnected according to an embodiment of the present invention.

FIG. 5 illustrates the overall communication diagram of the GUARDIAN system, depicting the interaction between the smart ring, mobile tower, cloud server, and trusted mobile devices.

FIG. 6 represents a use case scenario showing body vital and data synchronization within the geo-fencing area according to an embodiment of the present invention.

FIG. 7 depicts a use case scenario showing location tracking, notification triggers, and data synchronization outside the geo-fencing area according to an embodiment of the present invention.

FIG. 8 illustrates the voice communication scenario for emergency or need-based direct communication at the Smart Ring end according to an embodiment of the present invention.

### DETAILED DESCRIPTION OF THE FIGURES

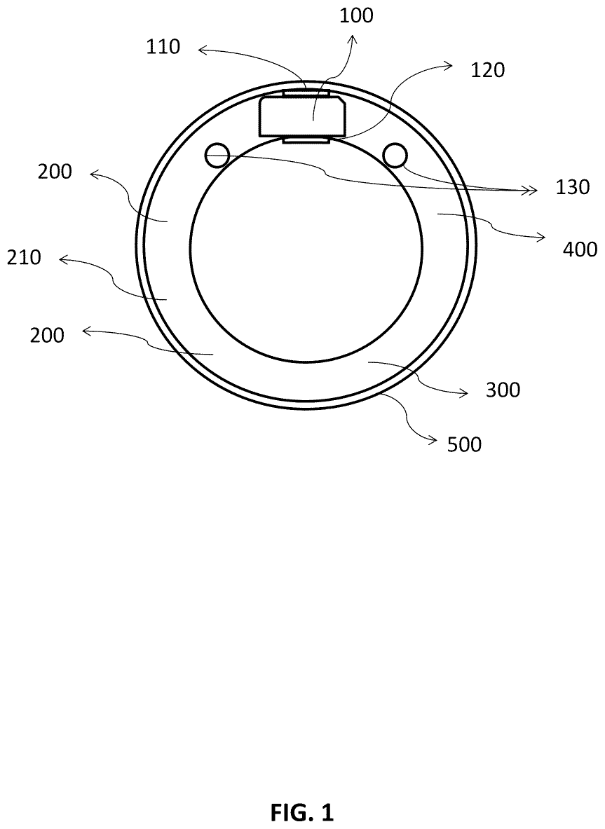

The GUARDIAN smart ring's basic configuration is illustrated in FIG. 1, which presents a circular arrangement of essential components designed to maximize functionality within the compact form factor. The device's architecture centers around a sophisticated integration of communication and health monitoring technologies. At the top of the ring, the Nano SIM/eSIM module 100 is positioned alongside the speaker unit 110 and microphone 120, forming the core communication cluster. These components are complemented by a tactile push/tap button 130 that provides user interface functionality. The ring's health monitoring capabilities are enabled by strategically placed heart rate sensors 200 and a G-Sensor 210, with an additional IR Sensor/Rx 220 enhancing the vital sign monitoring capabilities. A battery unit 300 and microprocessor 400 power and control these systems, all encased within an elastic, water-proof body 500 that ensures durability while maintaining user comfort.

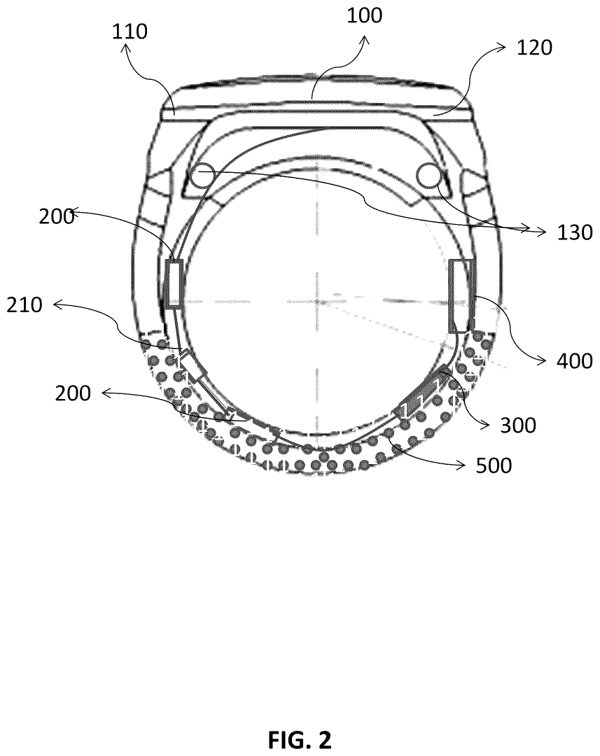

Moving to FIG. 2, a detailed cross-sectional view reveals the sophisticated layering of components within the GUARDIAN ring's structure. This view demonstrates how the device achieves its comprehensive functionality while maintaining a sleek profile. The upper section houses the communication components, including the Nano SIM/eSIM module 100, speaker 110, and microphone 120, arranged to optimize signal transmission and audio quality. The middle layer incorporates the push/tap button 130 and microprocessor 400, while the lower section contains the health monitoring sensors positioned for optimal contact with the user's finger. The entire assembly is protected by the water-proof enclosure 500, which provides both structural integrity and environmental protection.

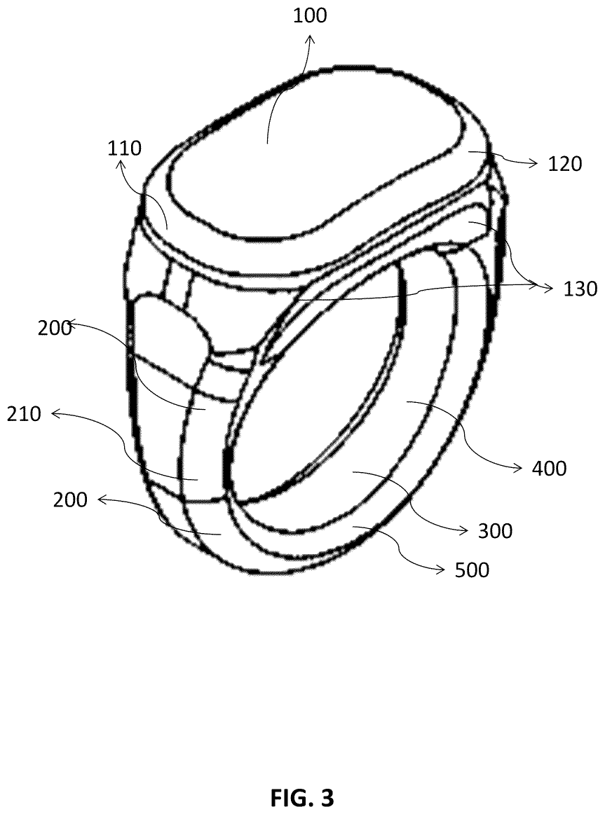

FIG. 3 presents a top-down perspective of the GUARDIAN ring, offering clear insight into the spatial relationship between components. This view highlights the ergonomic placement of the user interface elements and sensors, demonstrating how the design maximizes functionality while maintaining comfort and usability. The arrangement shows careful consideration of component placement to ensure reliable operation while minimizing interference between different systems.

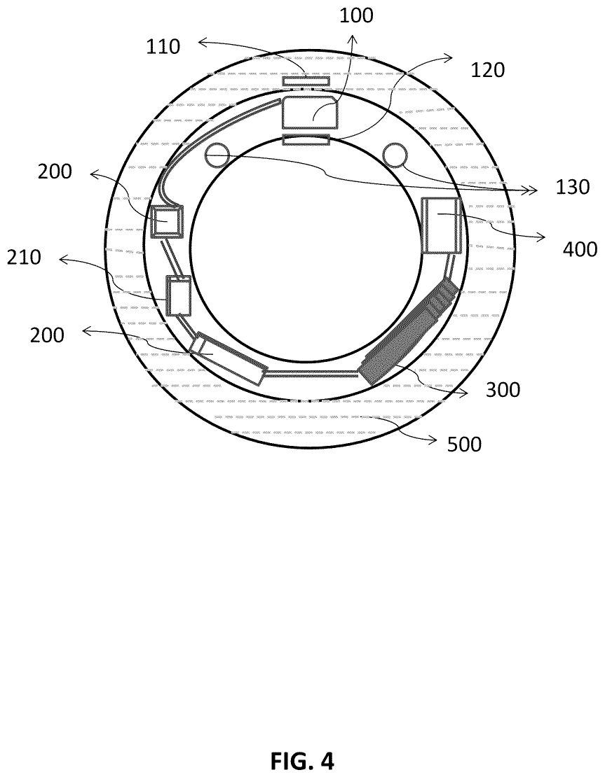

The interconnected nature of the GUARDIAN system's components is detailed in FIG. 4, which illustrates the sophisticated routing and connections between various elements. This figure demonstrates how the microprocessor 400 serves as the central hub, coordinating data flow between the communication modules, sensor array, and power management systems. The layout reveals the intricate balance between component density and functional efficiency, showing how each element is positioned to minimize signal interference while maintaining optimal performance.

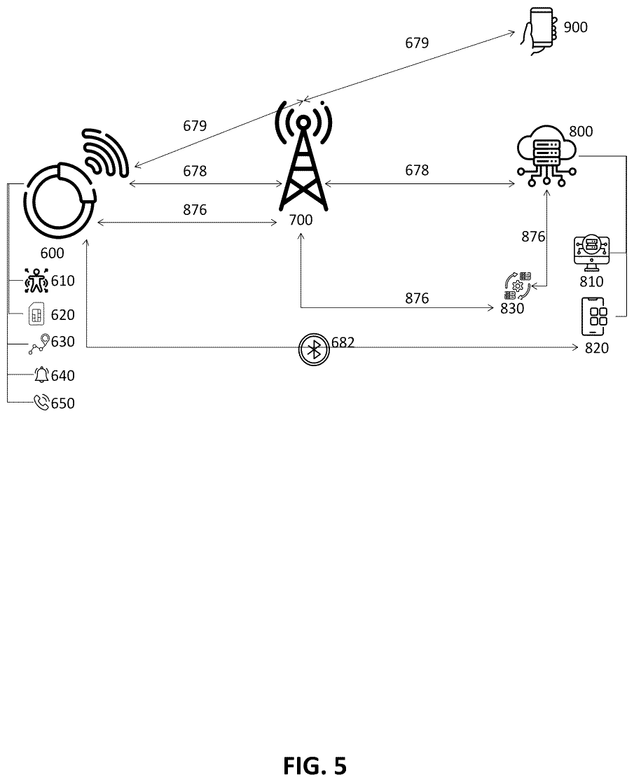

FIG. 5 presents the overarching communication architecture of the GUARDIAN system, showing how the smart ring interacts with external networks and devices. The diagram illustrates the multiple communication pathways established between the Wireless Smart Ring 600 and supporting infrastructure, including mobile towers 700, cloud-based servers 800, and trusted mobile devices 900. This comprehensive network enables features such as real-time health monitoring through body sensors 610, location tracking 630, and two-way voice communication 679, all managed through sophisticated data synchronization protocols 876.

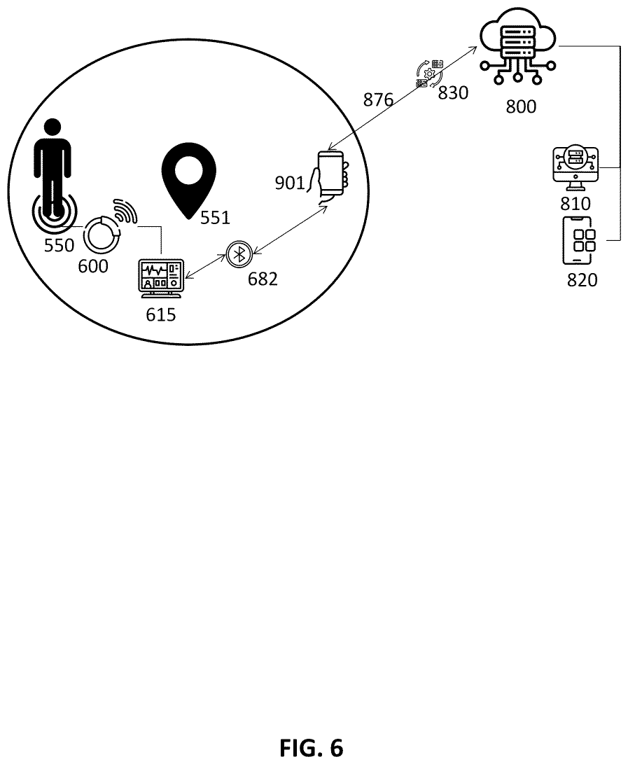

The system's behavior within a geo-fencing boundary is depicted in FIG. 6, showing how the GUARDIAN ring operates when the user remains within a defined area. This scenario demonstrates the device's ability to optimize power consumption and data transmission through intelligent use of Bluetooth connectivity 682 while maintaining continuous health monitoring and data synchronization with cloud services 800. The management application 810 processes incoming data while the customized mobile app 820 provides user interface functionality.

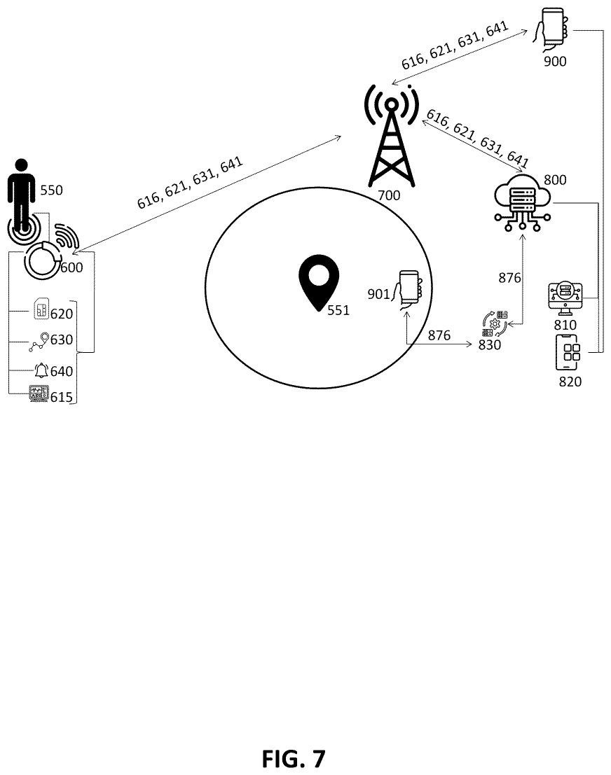

FIG. 7 illustrates the GUARDIAN system's operation when the user moves beyond the geo-fencing boundary, showcasing the device's ability to maintain functionality through cellular connectivity. This figure details how the ring transitions from local Bluetooth communication to cellular data transmission, ensuring continuous monitoring and communication capabilities regardless of the user's location. The diagram shows the interaction between various components including body sensors 615, location tracking 630, and notification systems 640, all working in concert to maintain service continuity.

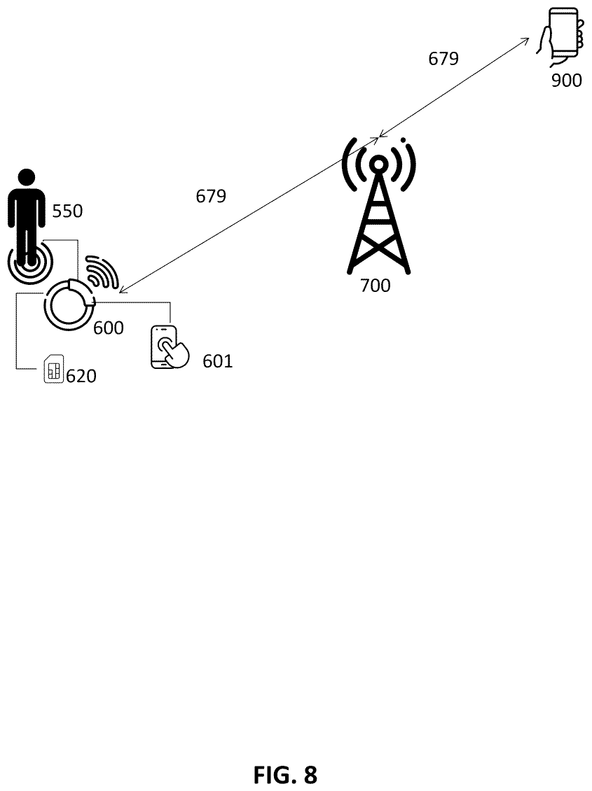

The emergency communication capabilities of the GUARDIAN system are illustrated in FIG. 8, which demonstrates the direct voice communication feature. This figure shows how users can initiate or receive calls through the smart ring's integrated soft push button 601 and cellular module 620, establishing immediate two-way voice communication 679 with trusted contacts 900 through mobile network infrastructure 700. This functionality operates independently of smartphone proximity, ensuring reliable communication in emergency situations.

Throughout all figures, special attention is given to demonstrating how the GUARDIAN system maintains its sophisticated functionality while adhering to size constraints and user comfort requirements. The illustrations collectively show how the device achieves its dual objectives of comprehensive health monitoring and independent communication capabilities within a wearable ring form factor.

What is claimed is:

1. A wearable smart ring device comprising: a ring-shaped housing having an adjustable and waterproof structure; a cellular communication module comprising a nano-SIM/eSIM system integrated within the housing;
a speaker and microphone embedded in the housing for two-way voice communication; a plurality of health monitoring sensors comprising a heart rate sensor, blood oxygen sensor, and fall detection sensor; a location tracking module for real-time position monitoring;
a microprocessor coupled to the cellular communication module and the health monitoring sensors;
a push/tap interface for user interaction and emergency activation; a battery power source; and
wherein the device is configured to operate independently of a smartphone while maintaining continuous cellular connectivity and health monitoring capabilities.

2. The wearable smart ring device of claim 1, wherein the health monitoring sensors further comprise a heart rate variability (HRV) sensor, a stress monitoring sensor, a sleep tracking sensor, an infrared sensor for vital sign monitoring, and an accelerometer for fall detection and motion analysis.

3. The wearable smart ring device of claim 1, wherein the ring-shaped housing comprises an elastic, waterproof outer shell, an adjustable band mechanism, a component housing section, and integrated antenna systems for cellular and location services.

4. The wearable smart ring device of claim 1, further comprising a geofencing module configured to detect device location relative to predefined boundaries, adjust operating modes based on location context, optimize power consumption based on location, and trigger automated responses based on geographical position.

5. The wearable smart ring device of claim 1, further comprising an artificial intelligence system configured to analyze health data patterns, detect anomalies in vital signs, process voice commands, recognize gestures, and trigger automated emergency responses.

6. A method of operating a smart ring communication and health monitoring system comprising: establishing independent cellular communication through an integrated nano-SIM/eSIM module within a wearable ring device; continuously monitoring user vital signs through a plurality of integrated health sensors, processing two-way voice calls through an embedded speaker and microphone system; tracking user location in real-time through a location monitoring module; detecting emergency situations through analysis of sensor data; automatically initiating emergency protocols upon detection of abnormal health patterns or manual emergency activation; synchronizing collected health and location data with a cloud server; and providing automated alerts to predefined emergency contacts including current user location and health status.

7. The method of claim 6, further comprising analyzing health data through artificial intelligence algorithms, generating predictive health alerts, managing power consumption based on usage patterns, implementing secure data transmission protocols, and maintaining encrypted communication channels.

8. The method of claim 6, wherein detecting emergency situations comprises monitoring accelerometer data for fall detection, analyzing vital signs for critical deviations, processing manual emergency trigger inputs, validating emergency conditions, and initiating appropriate response protocols.

9. A smart ring management system comprising: a wearable ring device having integrated cellular communication capabilities and health monitoring sensors; a cloud-based server infrastructure configured to process and store health data received from the ring device, manage real-time communication between the ring device and emergency services;
maintain user profiles and emergency contact information, and analyze health patterns through artificial intelligence algorithms; a management application running on the server configured to process incoming sensor data, trigger automated emergency responses, manage device configurations, and maintain communication logs; a mobile application providing user interface for viewing real-time health data, configuring device settings, managing emergency contacts, and accessing historical data; and wherein the system enables autonomous operation of the ring device while providing centralized management and emergency response capabilities.

10. The system of claim 9, wherein the cloud-based server infrastructure comprises data storage systems for health and location history, artificial intelligence processing engines, emergency response coordination systems, user profile management systems, and secure communication protocols.

11. The wearable smart ring device of claim 1, further comprising gesture recognition capabilities for device control, customizable alert patterns, adaptive power management, encryption systems, and biometric authentication.

12. The method of claim 6, further comprising detecting user presence within geofenced areas, switching between cellular and Bluetooth communication, optimizing power consumption, logging system events, and managing data privacy settings.

13. The system of claim 9, wherein the artificial intelligence algorithms are configured to learn user health patterns, identify potential health issues, customize alert thresholds, optimize device operation, and improve emergency response accuracy.

14. The wearable smart ring device of claim 1, wherein the cellular communication module supports automatic network switching, emergency calls without active service, two-way voice communication, data connectivity, and location services.

15. The method of claim 6, wherein monitoring vital signs comprises continuous heart rate tracking, blood oxygen level monitoring, stress level assessment, sleep pattern analysis, and motion tracking.

16. The system of claim 9, further comprising real-time health monitoring, automated emergency response, location tracking, data synchronization, and user notification management.

17. The wearable smart ring device of claim 1, comprising operating modes including normal monitoring mode, emergency mode, power-saving mode, geofence-based mode, and silent mode with vibration alerts only.

18. The method of claim 6, further comprising security measures including multi-factor authentication, encrypted communications, privacy controls, data access management, and regulatory compliance.

19. The system of claim 9, wherein the management application provides health analytics, automated reporting, emergency coordination, device monitoring, and system optimization.

20. The wearable smart ring device of claim 1, further comprising water resistance to IP68 standards, extended battery life capability, wireless charging capability, impact resistance, and an adjustable fit mechanism.

21. The method of claim 6, wherein emergency protocols comprise automated emergency contact notification, location data transmission, vital sign data sharing, two-way communication establishment, and emergency service coordination.
